(19) United States
(12) Patent Application Publication
(10) Pub. No.: US 2011/0288185 A1
(43) Pub. Date: Nov. 24, 2011

(54) LARGE SCALE GREEN MANUFACTURING OF METHANE USING PLASMA

(76) Inventor: James Charles Juranitch, Ft. Lauderdale, FL (US)

(21) Appl. No.: 12/998,693

(22) PCT Filed: Nov. 19, 2009
(86) PCT No.: PCT/US2009/006202

§ 371 Date: Aug. 8, 2011

Related U.S. Application Data
(60) Provisional application No. 61/199,760, filed on Nov. 19, 2008.

(51) Int. Cl. C07C 27/06

(52) U.S. Cl. 518/704

(57) ABSTRACT

A method and system for converting waste using plasma into methane. The method uses minimal fossil fuel, and therefore produces a minimal carbon footprint when compared to conventional processes. The method includes the steps of supplying a biomass material to a plasma melter; supplying electrical energy to the plasma melter; supplying steam to the plasma melter; extracting a syngas from the plasma melter; extracting hydrogen from the syngas; and forming methane from the hydrogen produced in the step of extracting hydrogen.

### RELATIONSHIP TO OTHER APPLICATION

This application claims the benefit of the filing date of U.S. Provisional Patent Application Ser. No. 61/199,760, filed on Nov. 19, 2008, Confirmation No. 1108 (Foreign Filing License granted). The disclosure in the identified provisional patent application is incorporated herein by reference.

### BACKGROUND OF THE INVENTION

1. Field of the Invention

This invention relates generally to methods and systems for extracting methane, and more particularly, to a system for manufacturing methane on a large scale from waste.

2. Description of the Related Art

In the current energy environment there is continuing pressure to produce more products and energy in a cost effective and clean way. Fuel prices continue to climb, and emission standards continue to tighten. Most of the modern world has attempted to limit the amount of carbon dioxide that is emitted into the atmosphere. It is considered by many that this gas has some responsibility in the climatic changes commonly referred to as global warming.

A current method of producing methane is to mine it from beneath the surface of the earth. All fossil fuel based methane that is burned produces additional greenhouse gasses. There is need for a methane generator the uses renewable resources, such as municipal waste. Such usage would render the methane that is produced to be carbon neutral. More specifically, when burned, the methane thus produced will not add any additional greenhouse gases to the atmosphere.

In current applications of plasma melters the product syngas is usually burned in a stationary power generator to make electricity. This is a reasonable use of the reclaimed power, but it is not the best use. Although small generating plants are inherently inefficient, the continuously varying BTU content of syngas renders this to be one of the best uses of mildly refined syngas.

There is a need in the art to redirect the syngas and convert it into a commodity that can widely be used in an efficient manner. Such a commodity is methane, commonly referred to as “natural gas.” Although methane differs slightly from the natural gas used by most people, the names have been used interchangeably in the art. This interchangeability is due to the fact that the BTU content, or energy level of methane, is essentially the same per cubic foot as the commonly used natural gas. This makes methane a very tradable commodity in today's energy markets. Additionally, methane can be used in optimized applications at very high levels of efficiency.

Plasma melters are now becoming a reliable technology that is used to destroy waste. At this time there are few operational plasma melter installations, but the technology is gaining acceptance. It is a characteristic of plasma melters that they produce a low BTU syngas consisting of several different elements. If the plasma melters are operated in a pyrolysis mode of operation, they will generate large amounts of hydrogen and carbon monoxide. The syngas byproduct is typically used to run stationary power generators. The resulting electric power is then sold to the power grid.

It is, therefore, an object of this invention to provide a system for liberating methane.

It is another object of this invention to provide a system for liberating methane on a large scale and that does not require large electrical generation resources.

It is also an object of this invention to provide a system for liberating methane that does not require consumption of natural resources.

It is a further object of this invention to provide a method and system of producing methane inexpensively.

It is additionally an object of this invention to provide an inexpensive method of using hydrogen to produce methane.

It is yet a further object of this invention to provide an inexpensive method of using a plasma melter to generate large amounts of methane.

It is also another object of this invention to provide a method of generating methane wherein waste carbon dioxide is obtained from a renewable energy source and therefore does not constitute an addition to the greenhouse gas carbon base.

### SUMMARY OF THE INVENTION

The foregoing and other objects are achieved by this invention which provides a method of manufacturing methane on a large scale. In accordance with the invention, the method includes the steps of:

supplying a waste material to a plasma melter;

supplying electrical energy to the plasma melter;

supplying steam to the plasma melter;

extracting a syngas from the plasma melter;

extracting hydrogen from the syngas; and

forming methane from the hydrogen produced in the step of extracting hydrogen.

In an advantageous embodiment of the invention, the waste material that is supplied to the plasma melter is a municipal waste. In other embodiments, the waste material is a municipal solid waste, and in still other embodiments the waste material is a biomass. In some embodiments where the waste material is a biomass, the biomass is specifically grown.

In one embodiment of the invention, the step of extracting hydrogen from the syngas includes, but is not limited to, the steps of:

subjecting the syngas to a water gas shift process to form a mixture hydrogen and carbon dioxide; and

extracting hydrogen from the mixture of hydrogen and carbon dioxide.

In a further embodiment, the step of extracting hydrogen from the mixture of hydrogen and carbon dioxide includes, but is not limited to, the step of subjecting the mixture of hydrogen and carbon dioxide to a pressure swing adsorption process. In some embodiments, the step of extracting hydrogen from the mixture of hydrogen and carbon dioxide includes, but is not limited to, the step of subjecting the mixture of hydrogen and carbon dioxide to a molecular sieve. In a further embodiment, the step of extracting hydrogen from the mixture hydrogen and carbon dioxide includes, but is not limited to, the step of subjecting the mixture of hydrogen and carbon dioxide to an aqueous ethanolamine solution. In yet another embodiment, prior to performing the step of subjecting the syngas to a water gas shift process to form a mixture of hydrogen and carbon dioxide there is provided the step of pre-treating the output of the plasma melter to perform a cleaning and separation of the syngas.

In accordance with an advantageous embodiment of the invention, the step of forming methane from the hydrogen produced in the step of extracting hydrogen includes, without limitation, the step of subjecting the hydrogen to a Sabatier Reactor process. In one embodiment, prior to performing the step of forming methane from the hydrogen produced in the step of extracting hydrogen there is provided the further step of optimizing the production of methane by correcting the molar ratio of carbon monoxide and hydrogen in the Sabatier Reactor process. The step of correcting the molar ratio of carbon monoxide and hydrogen in the Sabatier Reactor process includes, but is not limited to, the step of supplying a mixture of hydrogen and carbon monoxide to the Sabatier Reactor process.

In a further embodiment, there is provided the step of correcting the molar ratio of carbon dioxide and hydrogen in the Sabatier Reactor process. In this regard, hydrogen and carbon dioxide are diverted from the output of the water gas shift process, and are delivered to the Sabatier Reactor process.

In an advantageous embodiment of the invention, the step of supplying the mixture of hydrogen and carbon monoxide to the Sabatier Reactor process includes, but is not limited to, the step of diverting a portion of the hydrogen and carbon monoxide produced by the plasma melter. The step of diverting a portion of the hydrogen and carbon monoxide produced by the plasma melter is performed, in one embodiment, after performing a step of cleaning the hydrogen and carbon monoxide produced by the plasma melter.

In a further embodiment of the invention, the Sabatier Reactor is constructed of an aluminum-based ceramic foam, or other appropriately based foam.

In an advantageous embodiment of the invention, there is provided the step of extracting a slag from the plasma melter. In a further embodiment, the step of supplying a waste material to the plasma melter includes, but is not limited to, the step of supplying municipal waste to the plasma melter.

### BRIEF DESCRIPTION OF THE DRAWING

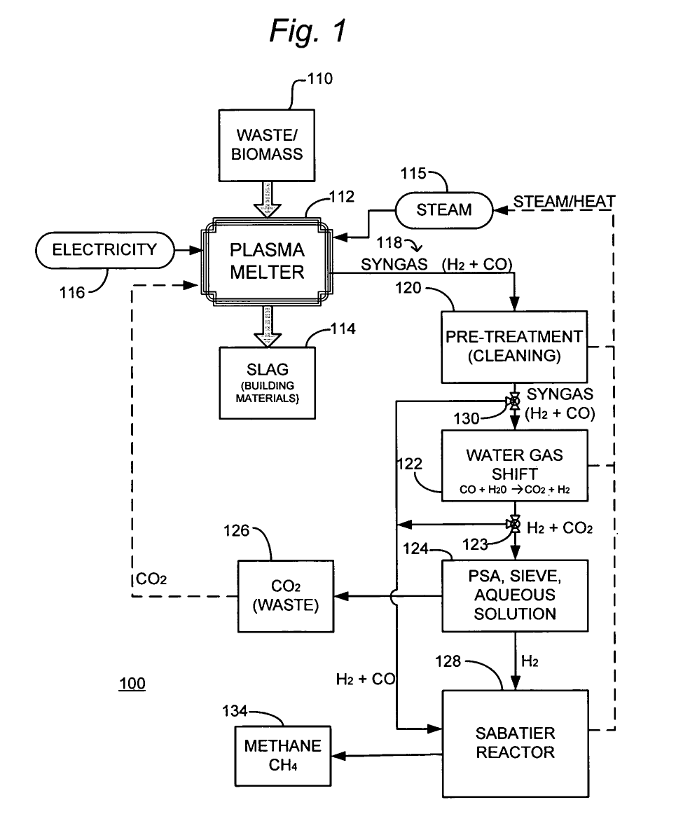

Comprehension of the invention is facilitated by reading the following detailed description, in conjunction with the annexed drawing, in which FIG. 1 is a simplified function block and schematic representation of a specific illustrative embodiment of the invention.

### DETAILED DESCRIPTION

FIG. 1 is a simplified function block and schematic representation of a specific illustrative embodiment of the invention. As shown in this FIGURE, a methane producing system 100 receives municipal waste, or specifically grown biomass 110 that is deposited into a plasma melter 112. In the practice of some embodiments of the invention, the process is operated in a pyrolysis mode (i.e., lacking oxygen). Steam 115 is delivered to plasma melter 112 to facilitate production of hydrogen and plasma. Also, electrical power 116 is delivered to plasma melter 112. A hydrogen rich Syngas 118 is produced at an output (not specifically designated) of plasma melter 112, as is a slag 114 that is subsequently removed.

In some applications of the invention, slag 114 is sold as building materials, and may take the form of mineral wool, reclaimed metals, and silicates, such as building blocks. In some embodiments of the invention, the BTU content, plasma production, and slag production can also be “sweetened” by the addition of small amounts of coke or other additives (not shown).

The syngas is cooled, cleaned, and separated in a pretreatment step 120. The carbon monoxide is processed out of the cleaned syngas at the output of a Water Gas Shift reaction 122. The waste carbon dioxide 126 that is later stripped out is not considered an addition to the greenhouse gas carbon base. This is due to the fact it is obtained in its entirety from a reclaimed and renewable source energy. In this embodiment of the invention, the energy source is predominantly municipal waste 110.

In some embodiments, the carbon dioxide is recycled into the plasma melter 112 and reprocessed into carbon monoxide and hydrogen. A Pressure Swing Adsorption (PSA) process, molecular sieve, aqueous ethanolamine solutions, or other processes are used in process step 124 to separate out carbon dioxide 126. Hydrogen from process step 124 is delivered to a conventional Sabatier Reactor 128, which is a known large scale process, or other similar process, to produce methane 134. In a highly advantageous embodiment of the invention, the Sabatier Reactor is formed of a ceramic foam that is based on aluminum or other appropriate material.

In this specific illustrative embodiment of the invention, a portion of the carbon monoxide and hydrogen obtained from pretreatment step 120 is diverted by a flow control valve 130 and supplied to Sabatier Reactor 128. This diverted flow is applied to achieve an appropriate molar ratio of carbon monoxide and hydrogen, and thereby optimize the production of methane. In addition, in this specific illustrative embodiment of the invention, a flow valve 123 diverts a portion of the hydrogen and carbon dioxide that is produced at the output of Water Gas Shift reaction 122 to Sabatier Reactor 128.

Pretreatment step 120, Water Gas Shift reaction 122, and Sabatier Reactor 128 generate heat that in some embodiments of the invention is used to supply steam to the plasma melter 112, or to a turbine generator (not shown), or any other process (not shown) that utilizes heat.

Although the invention has been described in terms of specific embodiments and applications, persons skilled in the art may, in light of this teaching, generate additional embodiments without exceeding the scope or departing from the spirit of the invention herein claimed. Accordingly, it is to be understood that the drawing and description in this disclosure are proffered to facilitate comprehension of the invention, and should not be construed to limit the scope thereof.

What is claimed is:

1. A method of manufacturing methane on a large scale, the method comprising the steps of:
supplying a waste material to a plasma melter;
supplying electrical energy to the plasma melter;
supplying steam to the plasma melter;
extracting a syngas from the plasma melter;
extracting hydrogen from the syngas; and
forming methane from the hydrogen produced in said step of extracting hydrogen.

2. The method of claim 1, wherein said step of extracting hydrogen from the syngas comprises the steps of:
subjecting the syngas to a water gas shift process to form a mixture of hydrogen and carbon dioxide; and
extracting hydrogen from the mixture of hydrogen and carbon dioxide.

3. The method of claim 2, wherein said step of extracting hydrogen from the mixture of hydrogen and carbon dioxide comprises the step of subjecting the mixture of hydrogen and carbon dioxide to a pressure swing adsorption process.

4. The method of claim 2, wherein said step of extracting hydrogen from the mixture of hydrogen and carbon dioxide comprises the step of subjecting the mixture of hydrogen and carbon dioxide to a molecular sieve.

5. The method of claim 2, wherein said step of extracting hydrogen from the mixture of hydrogen and carbon dioxide comprises the step of subjecting the mixture of hydrogen and carbon dioxide to an aqueous ethanolamine solution.

6. The method of claim 2, wherein prior to performing said step of subjecting the syngas to a water gas shift process to form a mixture of hydrogen and carbon dioxide there is provided the step of pre-treating the output of the plasma melter to perform a cleaning of the syngas.

7. The method of claim 1, wherein said step of forming methane from the hydrogen produced in said step of extracting hydrogen comprises the step of subjecting the hydrogen to a Sabatier Reactor process.

8. The method of claim 7, wherein the step of subjecting the hydrogen to a Sabatier Reactor process is performed in a Sabatier Reactor formed of a ceramic foam material.

9. The method of claim 8, wherein the ceramic foam material has an additive incorporated therein.

10. The method of claim 9, wherein the ceramic foam material is an aluminum based ceramic foam material.

11. The method of claim 7, wherein prior to performing said step of forming methane from the hydrogen produced in said step of extracting hydrogen there is provided the further step of optimizing the production of methane by correcting the molar ratio of carbon monoxide and hydrogen in the Sabatier Reactor process.

12. The method of claim 11, wherein said step of correcting the molar ratio of carbon monoxide and hydrogen in the Sabatier Reactor process comprises the step of supplying a mixture of hydrogen and carbon monoxide to the Sabatier Reactor process.

13. The method of claim 12, wherein said step of supplying the mixture of hydrogen and carbon monoxide to the Sabatier Reactor process comprises the step of diverting a portion of the hydrogen and carbon monoxide produced by the plasma melter.

14. The method of claim 13, wherein said step of diverting a portion of the hydrogen and carbon monoxide produced by the plasma melter is performed after performing a step of cleaning the hydrogen and carbon monoxide produced by the plasma melter.

15. The method of claim 7, wherein prior to performing said step of forming methane from the hydrogen produced in said step of extracting hydrogen there is provided the further step of optimizing the production of methane by correcting the molar ratio of carbon dioxide and hydrogen in the Sabatier Reactor process.

16. The method of claim 15, wherein said step of supplying the mixture of hydrogen and carbon dioxide to the Sabatier Reactor process comprises the step of diverting a portion of the hydrogen and carbon dioxide produced by a water gas shift process.

17. The method of claim 1, wherein there is further provided the step of extracting a slag from the plasma melter.

18. The method of claim 1, wherein said step of supplying a biomass material to the plasma melter comprises the step of supplying municipal waste to the plasma melter.

19. The method of claim 1, wherein the plasma melter is operated in a pyrolysis mode.
